(19) United States
(12) Patent Application Publication
(10) Pub. No.: US 2026/0023062 A1
(43) Pub. Date: Jan. 22, 2026

(54) METHOD AND SYSTEM FOR AUTOMATED AND CONTINUOUS MONITORING OF A FUNCTIONAL STATE OF A GAS MEASURING DEVICE

(71) Applicant: Dräger Safety AG & Co. KGaA, Lübeck (DE)

(72) Inventors: Malte Berndt, Lübeck (DE); Christof Rodehorst, Lübeck (DE); Raphael Maas, Lübeck (DE)

(21) Appl. No.: 19/273,451

(22) Filed: Jul. 18, 2025

(30) Foreign Application Priority Data
Jul. 19, 2024 (DE) 10-2024-120510.5

(51) Int. Cl. G01N 33/00

(52) U.S. Cl. CPC G01N 33/007

(57) ABSTRACT

The present disclosure relates to a method for the automated and continuous monitoring of a functional state of at least one gas measuring device. The method may comprise the following steps: generating of device characteristic values by the at least one gas measuring device, transmitting the device characteristics from the at least one gas measuring device to a data processing unit, determining a similarity measure of the device characteristic values, determining a functional state of the at least one gas measuring device based on the similarity measure and current device characteristics, generating a result value based on the functional state, and outputting the result value. Furthermore, the disclosure relates to a system for the automated and continuous monitoring of a functional state of at least one gas measuring device.

### CROSS-REFERENCE TO RELATED APPLICATIONS

This application claims the priority of German Patent Application No. 102024120510.5, filed on Jul. 19, 2024, and titled “METHOD AND SYSTEM FOR AUTOMATED AND CONTINUOUS MONITORING OF A FUNCTIONAL STATE OF A GAS MEASURING DEVICE”, which is hereby incorporated by reference in its entirety for all nonlimiting purposes.

### BACKGROUND

The present disclosure relates to a method and a system for automated and continuous monitoring of a functional state of at least one gas measuring device.

To monitor production facilities such as chemical or steel plants, refineries or mines, where toxic, explosive, oxygen-rich or oxygen-poor gas atmospheres can form in certain regions, gas measuring devices are used to protect the people in the regions and to protect the equipment. The gas measuring devices are equipped with suitable sensors to detect the concentration of gases or gas mixtures that are present in the respective region or that may occur in a dangerous concentration. Furthermore, the gas measuring devices have elements that provide a notification and/or alarm in the event of a concentration limit violation.

The gas measuring devices used in such production facilities must be checked at regular intervals to ensure they are ready for use. The checking of gas measuring devices is substantially carried out manually and usually by a qualified service technician or a responsible device maintenance person. In particular, it is checked in this case whether the gas measuring device is able to detect a dangerous gas concentration and generate a notification and/or an alarm, in particular in the event of a limit violation of a detected gas concentration. The check usually takes place on-site at the production facility, at the gas measuring device.

In order to check the functional state of gas measuring devices, the service technician or the device maintenance technician conventionally applies a suitable test gas to said devices, which they then check to see whether the gas measuring device reacts accordingly to the test gas and, in particular, generates a notification and/or an alarm.

Test devices or test stations are known, in particular for mobile gas measuring devices, which support the service technician or the device maintenance worker in the testing. The test devices or test stations offer, for example, the possibility of exposing the gas measuring device to a suitable test gas in order to trigger an alarm. The service technician or maintenance technician then evaluates the result and, if the test is positive (e.g., indicating no alarm is triggered), releases the gas measuring device for use.

Furthermore, test devices or test stations are known, in particular for mobile gas measuring devices, which apply a suitable test gas to the gas measuring device and detect whether an alarm or notification has been triggered, i.e. whether a dangerous gas concentration has been detected by the gas measuring device.

The aforementioned test devices or test stations are conventionally located in an equipment or service workshop where the gas measuring devices, in particular mobile gas measuring devices, are checked. The methods mentioned for checking gas measuring devices include at least some manual steps that must be carried out by a qualified service technician or a responsible device maintenance person. These manual steps result in time investment and correspondingly high costs, in particular in the case of production facilities with many gas measuring devices. Furthermore, these checks take place at longer intervals, for example monthly, weekly or daily depending on the field of application, so that a perfect functional state cannot be guaranteed between these intervals, since new functional errors of the gas measuring device can occur during this time. It would be conceivable to reduce the time intervals, but this would considerably increase the time required and the costs.

Typically, the checking of the gas measuring devices does not take place during their productive use, so alternative protective measures must be taken to monitor the production facility, or production may even have to be stopped for the duration of the check.

### SUMMARY

Proceeding from the prior art and the problems described above, the disclosure is based on the object of providing a technical solution in which the technical functionality of gas measuring devices is monitored particularly reliably and substantially continuously. In this case, the monitoring should be carried out automatically and at least largely without manual intervention and the productive operational capability of the gas measuring device should be maintained to protect people and systems.

The above objectives are achieved by a method for the automated and continuous monitoring of a functional state of at least one gas measuring device as described herein, and a system for the automated and continuous monitoring of a functional state of at least one gas measuring device as described herein. Further details of the disclosure emerge from the claims, the description, and the drawings. In this case, features and details which are described in connection with the method according to the disclosure also apply in connection with the system according to the disclosure, so that with regard to the disclosure of the individual aspects of the disclosure, reference is always made, or can be made, to each other.

### BRIEF DESCRIPTION OF THE DRAWINGS

In the figures:

FIG. 1 is an exemplary schematic view of an embodiment of the system according to the disclosure with one mobile and one stationary gas measuring device 1, 2;

FIG. 2 is an exemplary schematic view of an embodiment of the system according to the disclosure with one mobile gas measuring device 1; and

FIG. 3 is an exemplary schematic view of an embodiment of the system according to the disclosure with one mobile and two stationary gas measuring devices 1, 2, 30.

### DETAILED DESCRIPTION

The disclosure relates to a method for the automated and continuous monitoring of a functional state of at least one gas measuring device. The method comprises the following steps:
generating one or more device characteristic values using at least one gas measuring device,
transmitting the device characteristic values from the at least one gas measuring device to a data processing unit,
determining a similarity measure of the device characteristics, the similarity measure indicating a relationship between the device characteristics,
determining the functional state of the at least one gas measuring device on the basis of the similarity measure, in particular on the basis of the similarity measure and taking into account current device characteristics, and
generating a result value taking into account the functional state.

The functional state of at least one gas measuring device is understood to mean the technical functionality of the gas measuring device, i.e. the operative readiness of the gas measuring device or the operative readiness of one or more functionalities of the gas measuring device. The functionalities supported by a gas measuring device vary depending on the embodiment of the gas measuring device. However, the following functionalities are essential: detecting a concentration of a gas or a gas mixture, providing an alarm and/or notification in the event of a dangerous situation or an error condition, a dangerous situation existing, for example, if a limit value of a gas concentration is violated, and an error condition existing, for example, if a hardware component of the gas measuring device is defective.

A defect exists at least in cases where a hardware component fails technically or is at least temporarily in faulty, improper operation. At least, in this case, the hardware component is not operating as intended. Hardware components of a gas measuring device include, for example, light-emitting diodes, one or more loudspeakers and/or one or more vibration motors to signal an alarm, a screen and/or one or more sensors to measure environmental properties such as temperature or pressure, and one or more sensors to detect gases or gas mixtures and/or to measure their concentration in a monitoring region.

The at least one gas measuring device may be a mobile or a stationary gas measuring device. The mobile gas measuring device is portable and is usually attached to a person's clothing and/or personal protective equipment for personal protection. The mobile gas measuring device thus measures a gas concentration at different locations, depending on where the person is or where the mobile gas measuring device is currently located in the monitoring region, for example in a production facility. The stationary gas measuring device, in contrast, is fixed in one place and measures a gas concentration in a constant region of the production facility. Both types of gas measuring devices preferably include a communication unit for wireless and/or wired data exchange, for example with a central processing unit in the form of a control room, with a gateway or with a cloud.

In the first step of the method according to the disclosure, device characteristic values are generated by the gas measuring device. Device characteristic values may include, for example, gas measured values generated by one or more gas sensors, energy consumption values of a hardware component or a plurality of hardware components of the gas measuring device or of the gas measuring device itself, as well as measured values from environmental sensors, such as temperature, pressure or humidity. Furthermore, device characteristic values may also include state information of the gas measuring device, such as an alarm state, a normal state or a special state. The alarm state indicates, for example, that the gas measuring device has detected a violation of a gas concentration limit value. The normal state indicates, for example, that the gas measuring device is measuring and functioning as intended, and the special state indicates, for example, that the gas measuring device is not currently measuring any gas, e.g. if the gas measuring device is in a special operational mode, or has a defect. Other types of device characteristic values may be included within the scope of this disclosure.

In a next step, the device characteristic values are transferred from the gas measuring device to a data processing unit. The data processing unit is preferably a computing unit in a control room of the production facility, a computing unit of a gateway or, particularly preferably, a cloud, both data storage and data processing preferably taking place in the cloud. In this case, the data transmission takes place at least partly wired and/or at least partly wirelessly. The data processing unit preferably comprises at least one computer, a microprocessor, an FPGA (Field Programmable Gate Array) and/or comparable components for processing data.

Furthermore, in a next step, a similarity measure for the transmitted device characteristic values is determined by the data processing unit. A similarity measure is a means of indicating a relationship, in particular a stochastic dependence or stochastic independence, of a plurality of values to one another. A high degree of similarity exists, in particular for device characteristics of the same type, for example when the plurality of device characteristic values are substantially identical. Furthermore, there is a high degree of similarity, in particular in the case of device characteristics of different types, for example when the difference between pairs of values of the plurality of device characteristics is substantially identical to one another, i.e. for example in the case where a difference between pairs of values of the plurality of device characteristics recorded at the same time or in the same period is substantially identical. Furthermore, a high degree of similarity exists if device characteristic values generated in the same period of time behave similarly, for example if device characteristic values of a first gas measuring device increase and device characteristic values of another gas measuring device also increase in the same time period. In a concrete example, in this case measured values from a first temperature sensor of a first gas measuring device and measured values from a second temperature sensor of a second gas measuring device are considered similar if they rise or fall at the same time or in the same time period, i.e. if they behave in the same way.

Preferably, the similarity measure includes or takes into account probabilities of occurrence of specific device characteristics, in particular device characteristics of the same type, for example measured temperature values of a mobile gas measuring device. In this case, the probabilities of occurrence of the device characteristics are determined over a period of time, for example over one hour, several hours, one day or several days. In this case, the probabilities of occurrence indicate how often a particular device characteristic has been recorded within a recent period, for example how often a first specific temperature value and a second specific temperature value have been measured. In an advantageous manner, an evaluation of currently recorded device characteristics can be carried out on the basis of these probabilities of occurrence, which allows conclusions to be drawn about the functional state of the gas measuring device.

Furthermore, the similarity measure preferably includes or takes into account probabilities of occurrence of different device characteristics in dependence on one another, in particular device characteristics of different devices and/or different types, for example energy consumption values of an alarm signaling unit and gas sensor measured values of a gas measuring device. In this case, the probabilities of occurrence are also recorded over a period of time, as described above. Furthermore, in some examples the probabilities of occurrence may be predetermined, in particular in the case where there is a dependency relationship between the various device characteristics. For example, the energy consumption values of an alarm signaling unit increase when a limit value of the gas sensor readings is violated, since the alarm signaling unit is activated in this case. In an advantageous manner, an evaluation of currently recorded different device characteristics can be carried out on the basis of these probabilities of occurrence, which allows conclusions to be drawn about the functional state of the gas measuring device.

The determined similarity measure is thus used to compare the device characteristics and/or the corresponding device characteristic values with each other and shows how the device characteristics correlate. In this case, those types of device characteristics which normally exhibit a certain degree of similarity, i.e. an increased degree of similarity, and are correlated with each other, are particularly relevant. The normal case exists when the gas measuring device is operational, i.e. has no technical defect. This makes it possible to determine in an advantageous manner whether or at what point in time the previously similar and correlated device characteristics deviated from each other.

Based on the determined similarity measure and current device characteristics, the functional state of the gas measuring device is determined in a next step of the method according to the disclosure. The functional state indicates whether the gas measuring device is operational, has a defect or exhibits unusual behavior. In this case, it is determined whether the device characteristic values currently recorded by the gas measuring device and transmitted to the data processing unit correspond to the previously determined similarity measure, i.e. whether they have a substantially identical relationship to one another according to the determined similarity measure. When determining the functional state of the gas measuring device, it is preferable to take into account in particular those device characteristics which are normally highly similar to one another and strongly correlate with one another, the functional state of the gas measuring device being determined particularly reliably. If the current device characteristics do not align with the previously determined similarity measure, the data processing unit may determine that there is a defect or at least an unusual behavior of the gas measuring device. Preferably, the similarity measure includes or takes into account previously determined probabilities of occurrence of device characteristics, current device characteristics violating this similarity measure for example by falling below or exceeding a certain value of the probability of occurrence. If the current device characteristics substantially correspond to the determined similarity measure or if the current device characteristics behave in accordance with the similarity measure, then the data processing unit may assess the gas measuring device as operational.

In a further step of the method according to the disclosure, a result value is generated (e.g., by the processing unit) taking into account the determined functional state of the gas measuring device. The result value is preferably a digital signal that can be transmitted to a display unit or other output unit, e.g. an acoustical output unit, and/or to the gas measuring device, preferably a gas measuring device with a display or other output unit. The result value includes information about the technical functionality of the gas measuring device and/or a hardware component of the gas measuring device. In this case, the result value contains information about whether there is a defect or unusual behavior of the gas measuring device. Particularly preferably, the result value indicates whether an action is to be taken by a user of the gas measuring device. For example, whether the user should service or adjust the gas measuring device or whether the user should leave the region because the gas measuring device no longer provides protection for the user or only partially provides protection. In an advantageous manner, in this way the user of the gas measuring device has direct feedback and instructions for action, which increases his safety.

The particular advantage of the method according to the disclosure is the substantially continuous monitoring of the gas measuring device by comparing device characteristics and/or the behavior of device characteristics with each other, the monitoring being carried out automatically, in particular during the productive use of the gas measuring device. This means that the gas measuring devices are still operational and continue to perform their protective function, even during monitoring. This advantageously thus eliminates the need for additional expenditure on alternative protective measures or interruption of production during the inspection of the gas measuring device, or at least minimizes the effort involved while simultaneously improving safety.

Furthermore, the method is preferably fully automated so that manual steps by a person for checking the gas measuring device are not necessary. This advantageously also increases the reliability because, for example, errors due to incorrect checking of the gas measuring device are excluded, at least minimized or at least detectable at an early stage, and monitoring of a large number of technical functionalities of the gas measuring device as well as a large number of gas measuring devices is possible. The method according to the disclosure increases the safety for users of the gas measuring device and systems in a production facility whose environment is monitored by gas measuring devices.

In some examples, the device characteristics comprise at least energy consumption values of the at least one gas measuring device. In this case, the energy consumption values indicate, for example, the current power consumption and/or the electrical energy currently consumed. The energy consumption values relate to the gas measuring device and/or preferably to a corresponding hardware component of the gas measuring device, for example to a sensor, a signaling component such as a light-emitting diode, a loudspeaker and/or a vibration motor, to a computing unit such as a microprocessor, and/or to a display unit such as a screen.

During operation of a gas measuring device, the energy consumption values of a hardware component, for example the energy consumption values of a sensor or the energy consumption values of a computing unit, are often substantially identical and thus exhibit a high degree of similarity. If the energy consumption of such a hardware component decreases, for example at a certain point in time, at least an unusual behavior of the hardware component is detected, for example a warning is generated and a notification with this warning is sent to a user of the gas measuring device. In the event that the energy consumption falls below a threshold at which the hardware component is not functional according to the technical data sheet, the functional state is assessed as defective and preferably an error message is generated. In this case, corresponding information from technical data sheets of the relevant hardware components of the gas measuring device is preferably stored in the data processing unit or a data storage device that can be connected to the data processing unit.

Furthermore, energy consumption values of different hardware components of a gas measuring device are also suitable for determining a similarity measure and a functional state of the gas measuring device. Preferably, the similarity measure is determined for two or more signaling components, for example a similarity measure of the energy consumption values of a light-emitting diode and of a loudspeaker of the gas measuring device. These energy consumption values show a strong correlation with each other, as they are often activated simultaneously or at least partially with a time delay, the energy consumption increasing in each case when the light-emitting diode and the loudspeaker are activated. For example, if it is determined that only the energy consumption value of the light-emitting diode is increasing or has increased, and the energy consumption value of the loudspeaker is not, the functional state of the loudspeaker is assessed as defective, since in this case the energy consumption values are not similar to each other or do not correlate with each other. In this case, an error message is preferably generated as a result value, and transmitted to the gas measuring device, indicating this defective functional state. Another preferred example is the determination of a similarity measure of the energy consumption values of a vibration motor and the measured values of an acceleration sensor, the vibration motor and the acceleration sensor being included in a gas measuring device and the vibration motor being suitable for displaying a gas alarm or warning. The energy consumption values of the vibration motor and the measured values of the acceleration sensor are advantageously suitable because they should be similar to each other during normal operation of the gas measuring device. If the vibration motor is activated, its energy consumption thus increases and the vibration causes a change in the measured values of the acceleration sensor.

Advantageously, using the energy consumption values as one of the device characteristic values provide a reliable statement about the functional state of a gas measuring device and can also be determined with little effort.

According to one or more examples, the device characteristics comprise at least internal state information of the at least one gas measuring device. In this case, the internal state information indicates the mode that the gas measuring device is currently in. As previously described, this mode may preferably be a normal state, an alarm state, or a special state. For example, each state is assigned a specific value that is used to determine the similarity measure.

In a particularly advantageous manner, the internal state information of a gas measuring device can be linked to other types of device characteristics of the gas measuring device, a similarity measure being determined of at least one of the items of state information and another type of device characteristics, in particular an energy consumption value or a sensor measured value.

Particularly preferably, the similarity measure of the internal state information and the energy consumption of one or more signaling components is determined. In this case, the internal state information correlates with the energy consumption in such a way that the signaling components are inactive in the normal state and therefore do not consume any energy. In contrast, the signaling components are at least partially active in the alarm state and the energy consumption of at least one of the signaling components is measurable. If the behavior deviates from this, for example in the case that a signaling component of the gas measuring device does not consume any energy during the alarm state of a gas measuring device, the signaling component is assessed as faulty and the functional state of the gas measuring device as defective.

Furthermore, the similarity measure of the internal state information of two or more gas measuring devices that are located in spatial proximity to one another is preferably determined, the spatial proximity being determined via position data of the two or more gas measuring devices or via position data that are linked to the device characteristics.

The internal state information, in particular the normal state and the alarm state, of the two or more gas measuring devices located in close proximity to each other should be highly similar to each other, i.e. substantially identical, in particular in the case where the gas measuring devices comprise the same sensors. If it is determined that the items of internal state information have little or no similarity to each other, then at least one unusual behavior of the two or the one gas measuring device with different internal state information is determined or assumed and a warning is generated, the warning indicating this unusual behavior of the corresponding gas measuring devices or of the corresponding gas measuring device. For example, if the similarity measure is determined as a decimal value in the range from 0.0 to 1.0, where 1.0 indicates the highest similarity and 0.0 indicates the lowest or no similarity, the range of low similarity is preferably 0.1 to 0.5. Preferably, a similarity exists starting from a similarity measure of 0.6, particularly preferably from a similarity measure of 0.8.

In one or more examples, the device characteristics comprise at least sensor measured values of the at least one gas measuring device. In this case, the sensor measured values preferably include measured values from a temperature sensor, a humidity sensor, a pressure sensor and/or a gas sensor. At least some of the sensor measured values have a high degree of similarity to one another in the intended state of the at least one gas measuring device, so that these sensor measured values are advantageously suitable for evaluating a similarity.

Preferably, a similarity measure of sensor measured values of the same measured variable and different gas measuring devices is determined, for example the temperature measured values of a first gas measuring device with a first temperature sensor and of a second gas measuring device with a second temperature sensor. Furthermore, a similarity measure of sensor measured values of a single or different gas measuring devices is preferably determined, the corresponding sensor measured values having a technically determined dependency on one another. For example, the measured values of some gas sensors depend on pressure, temperature and/or humidity. In this case, the gas concentration value, in particular the raw value, i.e. the unprocessed measurement signal of the sensor, changes depending on the ambient pressure, the ambient temperature and/or the ambient humidity.

For example, the raw value of an electrochemical oxygen sensor increases when the pressure in the vicinity of the oxygen sensor increases. This behavior takes into account the determined similarity measure for sensor measured values of the gas sensor and sensor measured values of the pressure sensor. If this behavior does not occur, for example in the case that the raw value of the oxygen sensor remains substantially constant, although the pressure sensor measures an increased pressure at the same time, the functional state of the gas measuring device is determined to be at least abnormal or defective and at least a warning is generated.

Furthermore, certain gas sensors exhibit a dependency on each other, in particular gas sensors with cross-sensitivity. In this case, a gas sensor is considered cross-sensitive if its measured value changes due to a gas that is not the actual measuring gas of the gas sensor. For example, an electrochemical carbon monoxide sensor is sensitive to hydrogen, while its measuring gas is carbon monoxide. Such cross-sensitivity of a gas sensor can advantageously be taken into account when determining the similarity measure. For example, a gas measuring device includes an electrochemical carbon monoxide sensor and a hydrogen sensor. The similarity measure, which is determined for the measured values of the two sensors, takes into account the previously described cross-sensitivity of the carbon monoxide sensor and any deviating behavior is assessed as an abnormality or a defect of the gas measuring device and at least a warning is generated.

According to a preferred variant of the method, the similarity measure is determined by calculating the differences between device characteristics. In this case, the device characteristics are preferably of the same type. Particularly preferably, the similarity measure is determined on the basis of the sum of the absolute differences of the device characteristics. In this case, those device characteristics are highly similar to each other whose difference and/or sum of absolute differences is substantially zero or substantially steadily increasing, i.e. increasing at the same rate over time.

Advantageously, a similarity measure determined on the basis of a difference formation between the device characteristics can be implemented in a simple manner and requires a comparatively low computing power of the data processing unit.

In a preferred variant of the method, the similarity measure is determined on the basis of statistical correlations between device characteristics and is used as a prediction probability for expected device characteristics, the expected device characteristics being compared with the current device characteristics. In this case, a probability distribution of recorded device characteristics is determined and the functional state of the at least one gas measuring device is determined based on the probability distribution and current device characteristics. If current device characteristics are not probable or only probable to a small extent according to the probability distribution, then the at least one gas measuring device is exhibiting unusual behavior and a warning is generated that indicates this behavior of the relevant device characteristics, or indicates a hardware component related to the device characteristics. Particularly preferably, the similarity measure is determined using transinformation, also called mutual information, or normalized transinformation of device characteristics.

Advantageously, the previously described example of the method according to the disclosure provides a particularly reliable and accurate evaluation of the functional state of the at least one gas measuring device.

According to one or more examples, the similarity measure is determined for device characteristics of different types. In this case, dependencies on certain device characteristics of different types are advantageously taken into account when determining the functional state of at least one gas measuring device.

Information about such a dependency of certain device characteristics of different types on each other is preferably specified and is stored in the data processing unit or accessible to the data processing unit. For example, the previously described dependence of an electrochemical oxygen sensor on the ambient pressure is stored in the data processing unit.

Furthermore, in some examples the specific device characteristics and their dependencies may be determined by the data processing unit, with similarity measures being determined for a plurality of device characteristics over a longer period of time, for example several hours or days, and those device characteristics being used as specific device characteristics which have at least a certain similarity to one another over this period of time.

In one or more examples, the at least one gas measuring device comprises a first gas measuring device and at least one second gas measuring device. In this case, a similarity measure is determined for device characteristics of the first gas measuring device and the at least one second gas measuring device. Furthermore, the functional state of the first gas measuring device and/or of the at least one second gas measuring device is determined on the basis of this similarity measure and current device characteristics of the first gas measuring device and the at least one second gas measuring device.

In these examples, the similarity measure is thus determined on the basis of device characteristics of at least two different gas measuring devices. Preferably, the gas measuring devices have dependence on each other, for example two mobile gas measuring devices that are carried by one person and exposed to the same environmental conditions, or gas measuring devices that are located in a hall of a production facility. The similarity measure is determined according to one or more of the previously described variant embodiments. The functional state and the result value are determined for one or more gas measuring devices. For example, a similarity measure is determined for temperature measured values from three different stationary gas measuring devices located in a hall. The temperature measured values should be very similar to each other. In the event that the temperature measured values of one of the stationary gas measuring devices do not correspond to the determined similarity measure, at least an unusual behavior of this stationary gas measuring device is detected and a warning is generated indicating the unusual behavior.

By determining a similarity measure of device characteristics of different gas measuring devices, these examples advantageously offer even more comprehensive and reliable monitoring.

According to one or more examples, the method is characterized by the following additional steps:
transmitting position data of a location of the generation of the device characteristics of the at least one gas measuring device to the data processing unit
determining the similarity measure taking into account the position data assigned to the device characteristics.

The position data of the location where the device characteristics are generated are preferably generated by the at least one gas measuring device itself. In this case, the at least one gas measuring device preferably comprises a GPS receiver and/or similar components for determining a location.

In some examples, the position data of the location of the generation of the device characteristics may be generated by a separate external device. For example, the position data may be determined by an external GPS receiver and transmitted to the at least one gas measuring device. Further preferably, the position data are determined by a gateway and transmitted to the data processing unit, the at least one gas measuring device transmitting at least some of the device characteristics to the data processing unit via the gateway.

In some examples the position data are assigned to the at least one gas measuring device, preferably a stationary gas measuring device. In this case, the position data are stored, for example, in the data processing unit of the at least one gas measuring device. Furthermore, in some examples the position data are stored in a central processing unit, the at least one gas measuring device having access to the position data via a data interface.

The position data are preferably determined regularly, for example every second, and transmitted to the data processing unit, in particular in the case of mobile gas measuring devices. Particularly preferably, the position data are linked to the device characteristics and transmitted to the data processing unit together with the device characteristics.

Preferably, the similarity measure is determined for gas measuring devices that are spatially close to each other. For example, spatial proximity exists when two or more gas measuring devices are up to 10 m apart and/or are located together in a hall or in another specific region of the production facility.

Advantageously, the monitoring of at least one gas measuring device is even more efficient because position data can be used to determine which gas measuring devices are in close proximity to one another and thus, as expected, their device characteristics exhibit at least in part a high degree of similarity.

According to the disclosure, a system for the automated and continuous monitoring of a functional state of at least one gas measuring device by a data processing unit is further proposed. In this case, the at least one gas measuring device is configured to generate and transmit device characteristics, which may include at least energy consumption values, sensor measured values and/or internal state information of the gas measuring device. Furthermore, the data processing unit is configured to receive the device characteristics. The system according to the disclosure is characterized in that the data processing unit is configured to determine a similarity measure of the device characteristics, to determine the functional state of the at least one gas measuring device on the basis of the similarity measure and current device characteristics, and to generate a result value taking into account the functional state. In this case, the similarity measure indicates a relationship between the device characteristics.

As described above, the at least one gas measuring device is a mobile and/or stationary gas measuring device and the data processing unit is preferably a computing unit in a control room of the production facility, a computing unit of a gateway or particularly preferably a cloud, both data storage and data processing taking place in the cloud.

According to the method described herein or one of the previously described examples, the result value can be determined on the basis of the determined functional state of the at least one gas measuring device. In this case, the data processing unit is configured to determine the similarity measure using device characteristics of the at least one gas measuring device.

The similarity measure can be determined for device characteristics of the same type or for device characteristics of different types, of one or more gas measuring devices. Particularly preferably, the device characteristics and/or the plurality of gas measuring devices have a dependency on one another, so that at least some of their device characteristics are highly similar. If device characteristics deviate from the determined similarity measure, the data processing unit can detect at least one unusual behavior of the relevant gas measuring device, and a result value can be generated that indicates this unusual behavior.

Advantageously, the system according to the disclosure is designed to monitor at least one gas measuring device automatically and regularly and thus to protect persons or systems of a production facility, it being possible for the intended operation of the at least one gas measuring device to be determined and deviations from the intended operation to be detected.

In one or more examples of the system, the data processing unit is further configured to receive position data of a location of generation of the device characteristics of the at least one gas measuring device. In this case, the position data can be generated by the at least one gas measuring device or by a gateway. In some examples the data processing unit alternatively or additionally comprises position data of a location of the generation of the device characteristics of the at least one gas measuring device, in particular in the case of a fixed-location gas measuring device. Furthermore, the data processing unit is configured to determine the similarity measure taking into account the position data assigned to the device characteristics.

The at least one gas measuring device and/or the gateway preferably comprise a GPS receiver for determining the position data, the at least one gas measuring device and/or the gateway being configured to transmit the position data to the data processing unit preferably regularly, in particular every second, and/or together with the device characteristics.

Preferably, the similarity measure is determined, as described above, for device characteristics whose position data of the location of generation are in spatial proximity to one another, in particular for a plurality of gas measuring devices. Particularly preferably, the data processing unit is configured to determine the similarity measure for a plurality of gas measuring devices that are located at a distance of up to 10 m from one another and/or are located in a hall or in another specific area of the production facility.

Further features, objects and effects of the disclosure will become apparent from the following description of specific exemplary embodiments and from the accompanying figures. Exemplary embodiments of the disclosure are described without limiting the general inventive concept.

Exemplary embodiments of the invention are described in detail below with reference to the accompanying figures. Similar components in multiple figures are each provided with the same reference symbols.

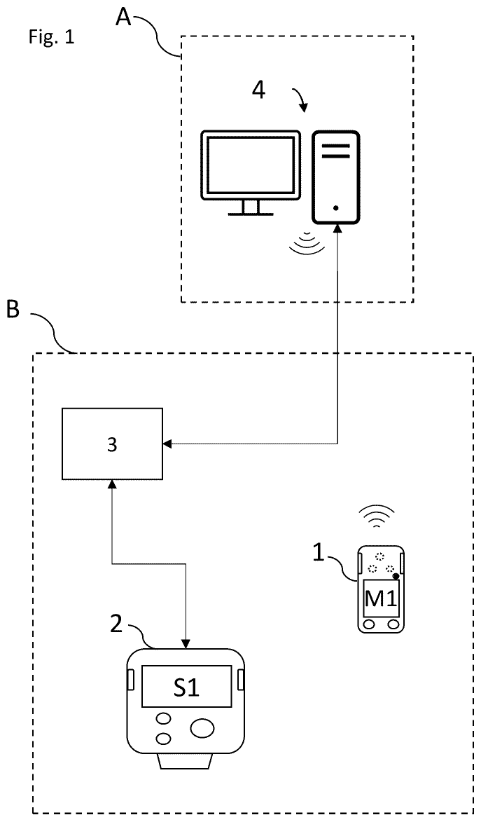

FIG. 1 shows an examples of a schematic view of an embodiment of the system according to the present disclosure with one mobile gas measuring device 1 and one stationary gas measuring device 2. The gas measuring devices are located within a production facility B. The mobile gas measuring device 1 comprises a communication interface for wireless communication and is configured to exchange data with a data processing unit via this communication interface. The stationary gas measuring device 2 comprises a communication interface for wired communication and is configured to exchange data with the data processing unit 4 via this communication interface. In this case, the data are first transmitted to a gateway 3 and then to the data processing unit 4 or to the stationary gas measuring device 2. The data processing unit 4 is located in a control room A, spatially separated from the region of the production facility B. The data processing unit comprises a processor, for example a microprocessor or another component for data processing, and a data memory.

The gas measuring devices 1, 2 generate a plurality of different device characteristics, in particular state information, gas measured values, temperature measured values and energy consumption values, the gas measuring devices 1, 2 being equipped with corresponding hardware components (e.g., gas measuring sensors, temperature measuring sensors, energy consumption sensors, and the like). The device characteristics are generated and transmitted to the data processing unit 4 every second. The data processing unit 4 determines a similarity measure for the relevant device characteristics. In this case, the device characteristics of a particular type, i.e. for the gas measured values of a corresponding sensor, for the temperature measured values, and for the energy consumption values, a probability of occurrence is determined and stored in the data memory of the data processing unit 4.

Following the determination of the corresponding similarity measure, the data processing unit evaluates the current device characteristics based on the associated similarity measure in each case.

If the current device characteristics show a high probability of occurrence according to the determined similarity measure, for example greater than 90%, then the gas measuring device 1, 2 is assessed as functional and a positive result value is generated. The result value is transmitted from the data processing unit to the gas measuring device 1, 2 and displayed in the form of, for example, a green LED on the gas measuring device 1, 2.

However, if the current device characteristics indicate a low probability of occurrence, for example less than 10%, then the gas measuring device 1, 2 and/or the corresponding component of the gas measuring device 1, 2 is assessed as defective. In this case, a negative result value is generated and transmitted from the data processing unit to the gas measuring device 1, 2, the gas measuring device 1, 2 displaying the negative result value in the form of, for example, a yellow LED and additionally displaying a corresponding note on a screen.

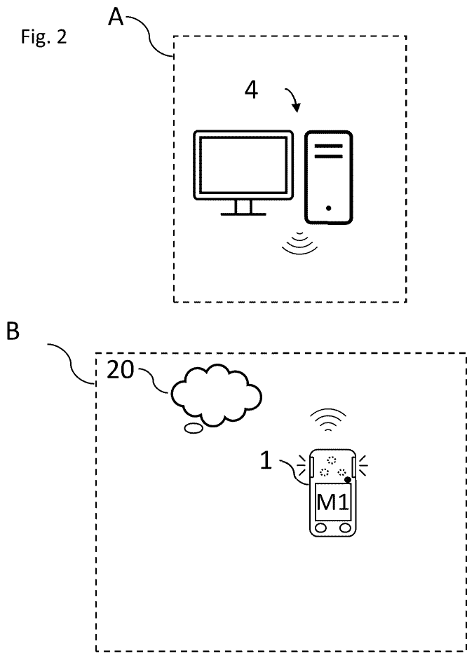

FIG. 2 shows an example of a schematic view of an embodiment of the system according to the invention with a mobile gas measuring device 1 and a data processing unit 4. The mobile gas measuring device and the data processing unit are designed and configured according to FIG. 1 to exchange data with each other.

The mobile gas measuring device 1 transmits state information and energy consumption values to the data processing unit 4, which determines a similarity measure based on these two device characteristic types. When the mobile gas measuring device 1 changes from the normal state to the alarm state, caused by an increased gas concentration of a gas cloud 20 in the vicinity of the mobile gas measuring device 1, the energy consumption values increase because the mobile gas measuring device emits an acoustic and/or visual alarm. The increase in energy consumption is taken into account in the determined similarity measure, the normal state being associated with low energy consumption values and the alarm state with higher energy consumption values.

If the similarity measure is violated when determining the functional state because the energy consumption values in the alarm state of the mobile gas measuring device 1 do not increase compared to the energy consumption values in the normal state, then a defect in the mobile gas measuring device 1 is detected and a negative result value is generated. In this case, the result value includes an indication of non-functional alarm elements of the mobile gas measuring device 1 and is transmitted to the mobile gas measuring device 1, which displays this indication in the form of an error message on the screen.

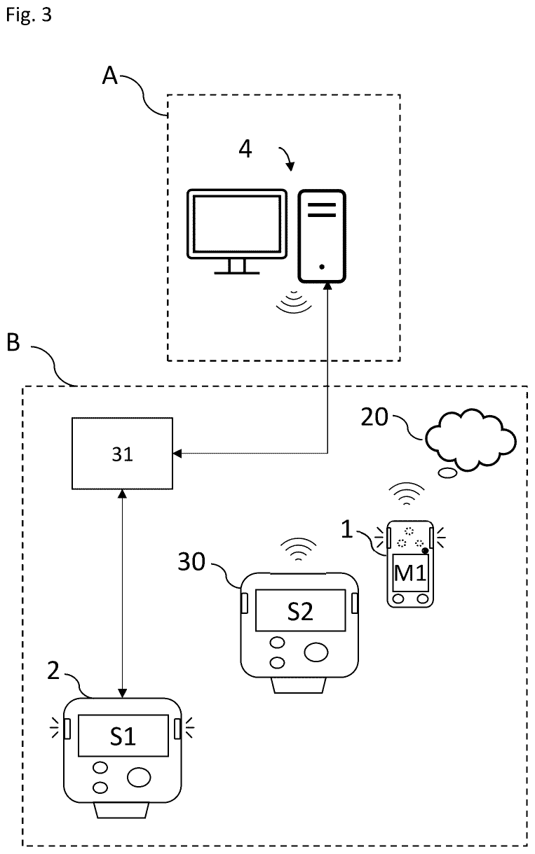

Based on FIG. 1, FIG. 3 is a schematic view of an example embodiment of the system according to the present disclosure with one mobile gas measuring device 1, one stationary gas measuring device 2 and a data processing unit 4. Accordingly, the description of FIG. 1 also applies to FIG. 3. Furthermore, FIG. 3 shows, in addition to FIG. 1, a second stationary gas measuring device 30, which comprises a communication interface for wireless communication and is configured to exchange data with the data processing unit 4. The gas measuring devices generate a plurality of device characteristics, including their state information, and transmit these to the data processing unit every second. Furthermore, the gas measuring devices 1, 2, 30 transmit position data of the location where the device characteristics are generated. For this purpose, gas measuring devices 1, 2, 30 each may include a GPS receiver. In some examples, the location data may be first transmitted to a second gateway 31 and then to the data processing unit 4 or to the stationary gas measuring device 2.

The data processing unit 4 in FIG. 3 generates a similarity measure based on the device characteristics of the gas measuring devices 1, 2, 30 taking into account the position data. In this case, the gas measuring devices 1, 2, 30 are arranged in spatial proximity to one another according to FIG. 3 and are spaced apart from one another by less than 10 m. According to the similarity measure determined, the state information of the gas measuring devices is substantially identical because they are located in spatial proximity to each other. The gas cloud 20 in FIG. 3 exceeds a limit value of a gas concentration permitted in the production facility B, which should be detected by the gas measuring devices 1, 2, 30. The mobile gas detector 1 and the stationary gas detector 2 detect this exceeding of the limit value, change from the normal state to the alarm state, and activate the signaling components, in particular optical signaling components, as indicated in FIG. 3 for the mobile and the stationary gas measuring device. The second stationary gas measuring device 30 does not detect this exceeding of the limit value because an inlet to a corresponding gas sensor is blocked. Accordingly, the second stationary gas measuring device remains in the normal state and does not switch to the alarm state.

The data processing unit 4 evaluates the current device characteristics on the basis of the determined similarity measure, only the state information of the mobile and the stationary gas measuring device 1, 2 being identical, and the state information of the second gas measuring device 30 differing therefrom. Consequently, the data processing unit generates a negative result value indicating a defective gas sensor and transmits it to the second stationary gas measuring device 30, which then displays a corresponding error message.

### LIST OF REFERENCE SIGNS

A control room
B production facility
1 mobile gas measuring device
2 stationary gas measuring device
3 first gateway
4 data processing unit
20 gas cloud
30 second stationary gas measuring device
31 second gateway

1.-11. (canceled)

12. A method for the automated monitoring of a functional state of at least one gas measuring device, comprising:
generating, by the at least one gas measuring device, a plurality of device characteristic values;
establishing, via a communication interface of the at least one gas measuring device, a connection between the at least one gas measuring device and a data processing unit;
transmitting the plurality of device characteristic values from the at least one gas measuring device to the data processing unit;
determining a similarity measure of the plurality of device characteristic values;
determining a functional state of the at least one gas measuring device based on the similarity measure and one or more current device characteristics;
generating, based on the functional state, a result value for the at least one gas measuring device; and
outputting the result value.

13. The method of claim 12, wherein the plurality of device characteristic values comprise one or more energy consumption values of the at least one gas measuring device.

14. The method of claim 12, wherein the plurality of device characteristic values comprise internal state information of the at least one gas measuring device.

15. The method of claim 12, wherein the plurality of device characteristic values comprise one or more sensor-measured values of the at least one gas measuring device.

16. The method of claim 12, wherein the determining the similarity measure comprises calculating differences between two or more device characteristic values of the plurality of device characteristic values.

17. The method of claim 12, wherein the determining the similarity measure is based on statistical relationships between two or more device characteristic values of the plurality of device characteristic values and wherein the similarity measure is used as a prediction probability for one or more expected device characteristics, the method further comprising:
comparing the one or more expected device characteristics with the one or more current device characteristics,
wherein the determining the functional state is further based on the comparing the one or more expected device characteristics with the one or more current device characteristics.

18. The method of claim 12, wherein the similarity measure comprises for a similarity between two or more device characteristic values of different types.

19. The method of claim 12, wherein the at least one gas measuring device comprises a first gas measuring device and at least one second gas measuring device and wherein:
the similarity measure indicates a similarity between one or more device characteristic values of the first gas measuring device and one or more device characteristic values of the at least one second gas measuring device and wherein the similarity measure is determined based on the one or more device characteristic values of the first gas measuring device and the one or more device characteristic values of the at least one second gas measuring device, and
at least one of the functional state of the first gas measuring device or a second functional state of the at least one second gas measuring device are determined based on the similarity measure and the one or more current device characteristics.

20. The method of claim 12, further comprising:
generating, using a GPS receiver, position data indicating a location corresponding to the generating of the plurality of device characteristic values; and
transmitting the position data to the data processing unit,
wherein the determining the similarity measure is further based on the position data.

21. The method of claim 12, wherein the outputting the result value comprises outputting the result value via a user interface of the at least one gas measuring device.

22. A system for the automated monitoring of a functional state of at least one gas measuring device, the system comprising:
a data processing unit comprising first memory storing first instructions that, when executed by the data processing unit, configure the data processing unit to monitor the at least one gas measuring device; and
the at least one gas measuring device, wherein the at least one gas measuring device comprises second memory storing second instructions that, when executed by one or more processors, configure the at least one gas measuring device to:
generate a plurality of device characteristic values corresponding to the at least one gas measuring device, wherein the plurality of device characteristic values comprise one or more of:
energy consumption values,
sensor measured values, or
internal state information of the at least one gas measuring device; and
establish, via a communication interface of the at least one gas measuring device, a connection between the at least one gas measuring device and the data processing unit,
wherein the first instructions, when executed by the data processing unit, further configure the data processing unit to:
receive, from the at least one gas measuring device, the plurality of device characteristic values;
determine a similarity measure of the plurality of device characteristic values;
determine a functional state of the at least one gas measuring device based on the similarity measure and one or more current device characteristics;
generate, based on the functional state, a result value; and
output the result value.

23. The system of claim 22, wherein the first instructions, when executed by the data processing unit, configure the data processing unit to determine the similarity measure by calculating differences between two or more device characteristic values of the plurality of device characteristic values.

24. The system of claim 22, wherein the first instructions, when executed by the data processing unit, configure the data processing unit to determine the similarity measure based on statistical relationships between two or more device characteristic values of the plurality of device characteristic values and wherein the similarity measure is used as a prediction probability for one or more expected device characteristics.

25. The system of claim 24, wherein the first instructions, when executed by the data processing unit, further configure the data processing unit to:
compare the one or more expected device characteristics with the one or more current device characteristics; and
wherein the determining the functional state is further based on the comparing the one or more expected device characteristics with the one or more current device characteristics.

26. The system of claim 22, wherein the similarity measure comprises for a similarity between two or more device characteristic values of different types.

27. The system of claim 22, wherein the first instructions, when executed by the data processing unit, further configure the data processing unit to:
receive position data indicating a location corresponding to the generating of the plurality of device characteristic values,
wherein the position data is generated by one or more of:
the at least one gas measuring device, or
a gateway, and
wherein first instructions, when executed by the data processing unit, configure the data processing unit to determine the similarity measure based on the position data.

28. The system of claim 27, wherein the at least one gas measuring device comprises a GPS receiver.

29. The system of claim 28, wherein the GPS receiver is separate from the at least one gas measuring device and from the data processing unit.

30. The system of claim 22, wherein the first instructions, when executed by the data processing unit, further configure the data processing unit to output the result value by causing output of the result value via a user interface of the at least one gas measuring device.

31. The system of claim 22, wherein the first instructions, when executed by the data processing unit, further configure the data processing unit to output the result value with one or more of:
an audio alert, or
a visual alert.
